(19) United States
(12) Patent Application Publication
(10) Pub. No.: US 2015/0258324 A1
(43) Pub. Date: Sep. 17, 2015

(54) TUBE CONNECTION STRUCTURE

(71) Applicant: TERUMO KABUSHIKI KAISHA, Tokyo (JP)

(72) Inventors: Takahiro CHIDA, Kawasaki-shi (JP); Kyouta KIMURA, Tokyo (JP); Youichirou KUWANO, Atsugi-shi (JP)

(73) Assignee: TERUMO KABUSHIKI KAISHA, Tokyo (JP)

(21) Appl. No.: 14/666,579

(22) Filed: Mar. 24, 2015

Related U.S. Application Data
(63) Continuation of application No. PCT/JP2014/000401, filed on Jan. 27, 2014.

(30) Foreign Application Priority Data
Jan. 28, 2013 (JP) 2013-013670

(51) Int. Cl. A61M 39/10

(52) U.S. Cl. CPC A61M 39/10; A61M 2039/1038

(57) ABSTRACT

A tube connection structure is disclosed, which includes a first connector and a second connector that are fitted together. The first connector includes an outer cylinder; a communicating section which is formed with a liquid passage in which a liquid can circulate in a fitted state, and an annular valve body which is provided around the communicating section and seals an outer cylinder opening portion in a non-fitted state. The second connector includes an inner cylinder which pushes the annular valve body in the fitted state, an inner valve body which seals an inner cylinder opening portion in the non-fitted state and is pushed by the communicating section advancing into the inner cylinder through the inner cylinder opening portion in the fitted state, and a first seal member which is provided at least on an inner peripheral surface of the inner cylinder opening portion.

### CROSS-REFERENCES TO RELATED APPLICATIONS

This application is a continuation of International Application No. PCT/JP2014/000401 filed on Jan. 27, 2014, and claims priority to Japanese Application No. 2013-013670 filed on Jan. 28, 2013, the entire content of both of which is incorporated herein by reference.

### TECHNICAL FIELD

The present disclosure generally relates to a tube connection structure which is used for connecting two tubes, for example, a tube connection structure which is suitably used for connecting a tube indwelled in the body of a patient, with a tube connected to various medical instruments or the like.

### BACKGROUND DISCUSSION

In the medical field, in some cases, various medical instruments are used by being connected to a tube which is indwelled in the body of a patient in order to maintain and manage the health of a patient. Specifically, for a patient who has difficulty in urinating by his or her own intention due to, for example, decreased function in the urinary organs and strength of muscles around the urinary organs, a so-called urine collection bag and a urethral catheter of which one end is indwelled by being inserted into the bladder from the urethra of the patient are connected for use. Urine discharged from the body of a patient is accumulated in the urine collection bag, which is connected to the other end of the urethral catheter.

In general, such a urine collection bag cannot be used by being disconnected from a patient since one end of the urethral catheter is indwelled in the bladder of the patient. Accordingly, the patient is forced to move together with the urine collection bag, which is connected to the bladder through the urethral catheter even during so-called rehabilitation, bathing, or the like. Accordingly, there is a concern that the patient using the urine collection bag may get a urinary tract infection, which can be developed by urine flowing backward in the bladder depending on the position or the condition of the urine collection bag.

For this reason, when using such a urine collection bag, it can be desirable to provide a tube connection structure configured to have a first connector and a second connector which are detachable in the middle of a urethral catheter so that a patient can be moved when the urine collection bag is temporarily disconnected.

Examples of the tube connection structure configured to have the first connector and the second connector which are detachable in this way include International Publication No. WO 2007/014281. In the tube connection structure in International Publication No. WO 2007/014281, as shown in FIG. 8, which is a cross-sectional view along an axial direction of a tube connection structure, a first connector 100 includes a first connection section 101 which is connected to one tube (not shown) at one end (right side in the drawing) in the axial direction, an outer cylinder 102 which is positioned further toward the other end (left side in the drawing) in the axial direction than the first connection section 101, a communicating tube 103 which extends to the outer cylinder 102 in the axial direction in the outer cylinder 102 and is formed with a liquid passage in which a liquid can circulate, an annular valve body 104 which is provided around the communicating tube 103 so as to be movable in the axial direction, and a first coil spring 105 that is provided by surrounding the communicating tube 103 and biases the annular valve body 104 to the other end in the axial direction. In addition, a second connector 150 can include a second connection section 151, which is connected to another tube which is not shown in the drawing, an inner cylinder 152 which is positioned further toward the one end in the axial direction than the second connection section 151 and pushes the annular valve body 104 to the one end in the axial direction by entering the outer cylinder 102, an inner valve body 153 which is provided in the inner cylinder 152, and a second coil spring 154, which can bias the inner valve body 153 to the one end in the axial direction.

According to the tube connection structure, a liquid can circulate within the tube connection structure as shown in FIG. 8 when the first connector 100 in the axial direction and the second connector 150 are connected to each other. In contrast, when the first connector 100 and the second connector 150 are not connected to each other, the other end of the first connector 100 in the axial direction is liquid-tightly sealed by the annular valve body 104 which is biased by the first coil spring 105, and the one end of the second connector 150 in the axial direction is liquid-tightly sealed by the inner valve body 153 which is biased by the second coil spring 154. Accordingly, with the tube connection structure in the middle of a urethral catheter, a patient can move in a state of being disconnected from a urine collection bag by separating the first connector and the second connector.

However, in a case where the tube connection structure, such as a urethral catheter, used for a living body is constituted by a first connector and a second connector so as to be separable from each other, it is necessary to sterilize the outer surface of the two connectors in advance in view of preventing the occurrence of infectious diseases when the connectors in a state of being separated from each other are connected to each other.

However, in the tube connection structure in shown in FIG. 8 of International Publication No. WO 2007/014281, it is necessary to liquid-tightly seal the abutting portion between the inner cylinder 152 and the annular valve body 104 in order to prevent leakage of a liquid from the inside of the tube connection structure, and in order to make the liquid circulate in the tube connection structure by pushing the annular valve body 104 of the first connector 100 in the inner cylinder 152 of the second connector 150. In addition, in the tube connection structure, in order to secure the sealing in the abutting portion between the annular valve body 104 and the inner cylinder 152 in the state where the first connector 100 and the second connector 150 are connected to each other, an annular convex portion 104a which protrudes from an outer surface of the annular valve body 104 is provided on the outer surface thereof and an annular concave portion 152b which is fitted with the annular convex portion 104a is provided on an outer surface of an inward flange portion 152a of the inner cylinder 152 which faces the outer surface of the annular valve body 104.

Accordingly, for this reason, in the tube connection structure in the aforesaid related art, it can be difficult to wipe the inside of the annular concave portion 152b recessed from the outer surface using, for example, a cloth soaked in drug when sterilizing the outer surface before connecting the first connector 100 and the second connector 150 to each other, which is an indispensable process in the tube connection structure used for a living body.

In addition, for example, when the first connector can be detached from the second connector and a connected state and non-connected state of these connectors are repeatedly used, it can be necessary to remove a liquid from the outer surface, on which the liquid flowing in a tube is adhered, after detaching the second connector from the first connector. However, in the tube connection structure shown in FIG. 8, it can be impossible to sufficiently wipe the liquid, which has entered the annular concave portion 152b.

Accordingly, there are problems such as an increased occurrence of urinary tract infections and other sanitary problems due to proliferation of bacteria or the like on the outer surfaces of the first connector 100 and the second connector 150 when using the tube connection structure in the related art as a tube connection structure used for a living body.

Moreover, in the tube connection structure shown in FIG. 8, in a non-connected state between the first connector 100 and the second connector 150 after the second connector 150 is detached from the first connector 100, it is particularly difficult to sterilize and remove a liquid from the outer surface of the annular valve body 104 since the annular valve body 104 of the first connector 100 is positioned very deeply inside (one end in the axial direction) the outer cylinder 102 from the opening portion 102a at the other end of the outer cylinder 102 in the axial direction as shown in FIG. 9.

The present disclosure has been made in order to solve the problems existing in the related art, and can provide a tube connection structure which can be relatively easily and reliably sterilized or the like of the first connector and the second connector through application of a drug before connecting the connectors to each other and can be used in sanitary conditions.

### SUMMARY

In accordance with an exemplary embodiment, a tube connection structure is disclosed, which can be used in a living body is constituted by a first connector which has a first connection section connected to a tube in one end portion in an axial direction; and a second connector which has a second connection section connected to a tube in the other end portion in the axial direction and of which the one end portion in the axial direction is fitted with the other end portion of the first connector in the axial direction. In a fitted state between the first connector and the second connector, a liquid can circulate between one end of the first connector in the axial direction and the other end of the second connector in the axial direction through the inside of the fitted first and second connectors, and in a non-fitted state between the first connector and the second connector, leakage of a liquid from the inside of the first connector to the other end of the first connector in the axial direction and leakage of a liquid from the inside of the second connector to one end of the second connector in the axial direction are prevented. The first connector can include an outer cylinder which is positioned further toward the other end in the axial direction than the first connection section; a communicating section which extends from the one end in the axial direction to an outer cylinder opening portion at the other end in the axial direction in the outer cylinder and is formed with a liquid passage in which the liquid can circulate in the fitted state; and an annular valve body which is provided around the communicating section so as to be movable in the axial direction and seals the outer cylinder opening portion at the other end of the outer cylinder in the axial direction in the non-fitted state. The second connector can include an inner cylinder, which is positioned further toward the one end in the axial direction than the second connection section and pushes the annular valve body to one side in the axial direction by entering the outer cylinder in the fitted state; an inner valve body which is provided in the inner cylinder so as to be movable in the axial direction, seals one end of the inner cylinder in the axial direction by being fitted with an inner cylinder opening portion of the one end of the inner cylinder in the axial direction in the non-fitted state and is pushed to the other end in the axial direction by the communicating section of the first connector advancing into the inner cylinder through the inner cylinder opening portion in the fitted state; and a first seal member which is provided at least on an inner peripheral surface of the inner cylinder opening portion and liquid-tightly seals the space between an outer peripheral surface of the communicating section of the first connector which is penetrated through the inner cylinder opening portion and the inner peripheral surface of the inner cylinder opening portion in the fitted state. The liquid passage of the communicating section is provided in a portion entering the inner cylinder in the fitted state.

In accordance with an exemplary embodiment, in the tube connection structure of the present disclosure, the inner cylinder can have a decreased diameter portion, which is provided with the inner cylinder opening portion and of which the inner diameter is decreased, in the one end portion in the axial direction, and that the first seal member extends from the inner peripheral surface of the inner cylinder opening portion to an inner surface side of the decreased diameter portion.

In addition, in the tube connection structure of the present disclosure, the inner diameter of the decreased diameter portion can gradually decrease from the other end in the axial direction to the one end in the axial direction, and the first seal member can extend to the other end of the decreased diameter portion in the axial direction.

Furthermore, in the tube connection structure of the present disclosure, the first seal member can extend onto an end surface at the one end of the inner cylinder in the axial direction.

Furthermore, in the tube connection structure of the present disclosure, an annular second seal portion can be provided on at least one of opposing surfaces counter to the annular valve body and the inner cylinder, so as to protrude from the one opposing surface and abut on the other opposing surface in the fitted state.

In accordance with an exemplary embodiment, in the tube connection structure of the present disclosure, the first connector further can include a first elastic body which biases the annular valve body toward the other end of the outer cylinder in the axial direction in the outer cylinder, and the second connector further can include a second elastic body which biases the inner valve body toward one side in the axial direction of the inner cylinder.

According to the tube connection structure of the present disclosure, the liquid passage of the communicating section of the first connector is provided in a portion penetrating the inner cylinder opening portion of the second connector and entering the inner cylinder in a fitted state of the first connector and the second connector, and the first seal member is provided at least on the inner peripheral surface of the inner cylinder opening portion and liquid-tightly seals the space between the outer peripheral surface of the communicating section of the first connector which is penetrated through the inner cylinder opening portion and the inner peripheral surface of the inner cylinder opening portion in the fitted state. Therefore, leakage from the inside of the tube connection structure can be prevented, while making a liquid circulate between one end of the first connector in the axial direction and the other end of the second connector in the axial direction in the fitted state.

In addition, in the tube connection structure of the present disclosure, leakage of a liquid can be prevented by using the first seal member in the fitted state, and therefore, it is unnecessary to form a concave portion, which is used for preventing leakage of a liquid in the fitted state, on an end surface of the first connector and an end surface of the second connector. Accordingly, the sterilization of these end surfaces can be performed relatively easily and reliably by wiping off a liquid or a foreign substance adhered on the end surfaces.

As a result, the tube connection structure of this disclosure can be used in sanitary conditions when the first connector and the second connector are connected to each other.

### BRIEF DESCRIPTION OF THE DRAWINGS

FIG. 1 is a cross-sectional view, along an axial direction of a tube connection structure, which shows an exemplary embodiment of a tube connection structure of the present disclosure in a state after a first connector and a second connector are fitted together.

FIG. 2 is a cross-sectional view, along the axial direction of the tube connection structure, which shows the tube connection structure of FIG. 1 in a state before the first connector and the second connector are fitted together.

FIG. 3A is an enlarged cross-sectional view, along the axial direction of the tube connection structure, which shows a main section of an inner cylinder of the second connector of the tube connection structure of FIG. 1.

FIG. 3B is an enlarged cross-sectional view, along the axial direction of the tube connection structure, which shows an end portion of the first connector of the tube connection structure of FIG. 1 on a side which is connected to the second connector in a state before the first connector and the second connector are fitted together.

FIG. 4 is a perspective view showing each of constituent members of the first connector of the tube connection structure of FIG. 1 by decomposing the first connector.

FIG. 5 is a perspective view showing each of constituent members of the second connector of the tube connection structure of FIG. 1 by decomposing the second connector.

FIG. 6 is an enlarged cross-sectional view, along the axial direction of the tube connection structure, which shows a modification example of a formation mode of a second seal member of the tube connection structure of FIG. 1 in a state after the first connector and the second connector are fitted together.

FIG. 7A is a view of an outer peripheral surface of the inner cylinder of the second connector of the tube connection structure of FIG. 1, which shows the shape of a guide groove portion provided in the inner cylinder.

FIG. 7B is a view of an outer peripheral surface of the inner cylinder, which shows the shapes of guide groove portions of an exemplary example.

FIG. 7C is a view of an outer peripheral surface of the inner cylinder, which shows the shapes of guide groove portions of an exemplary example.

FIG. 8 is a cross-sectional view along an axial direction of a tube connection structure in the related art.

FIG. 9 is a cross-sectional view, along the axial direction of the tube connection structure, which shows the first connector of the tube connection structure of FIG. 8 in a state of being detached from the second connector.

### DETAILED DESCRIPTION

Hereinafter, an embodiment of the disclosure will be described with reference to the accompanying drawings.

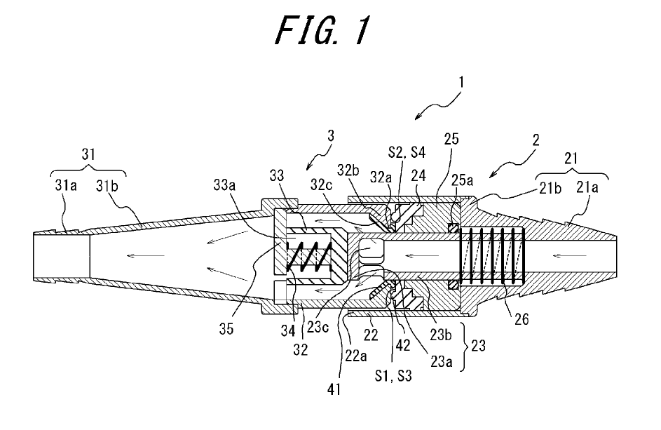

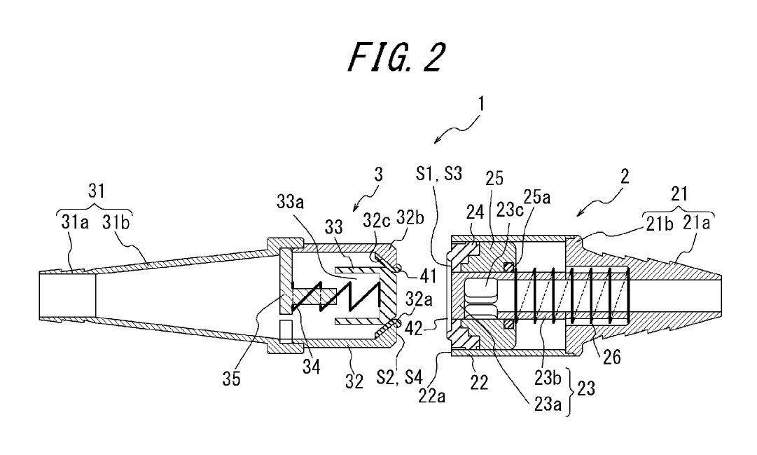

A tube connection structure 1 shown in FIGS. 1 and 2 is configured to have a first connector 2 and a second connector 3 which is attached to the first connector 2, and can be used, for example, when connecting two tubes to each other which constitute a urethral catheter that guides urine, discharged from the body of a patient, from the bladder to a urine collection bag.

Here, as shown in FIGS. 1 and 2 which are cross-sectional views along an axial direction (horizontal direction in FIG. 1, in which the right side thereof is one end in the axial direction and the left side thereof is the other end in the axial direction) of the tube connection structure 1, the first connector 2 has a first connection section 21, which is connected to an end portion of one tube between two tubes which are not shown in the drawings, in one end portion in the axial direction of the first connector 2. In addition, the second connector 3 has a second connection section 31, which is connected to an end portion of the other tube, in the other end portion in the axial direction of the second connector 3.

In addition, the view shown in FIG. 1 is in a fitted state where one end portion in the axial direction of the second connector 3 is fitted with the other end portion in the axial direction of the first connector 2, and the view shown in FIG. 2 is in a non-fitted state where the second connector 3 is detached from the first connector 2.

In accordance with an exemplary embodiment, a tube connected to the first connector 2 and the second connector 3 is not limited to the urethral catheter, and may be a medical and welfare tube of which one end is indwelled in the body of a patient and in which, for example, blood, abdominal dropsy or pleural effusion, bile, gastrointestinal tract fluids, operative field leachate, operative field cleaning solutions, drainage solutions of cerebrospinal fluids, peritoneal dialysis fluids, and other liquids flow.

In accordance with an exemplary embodiment, the first connector 2 shown in FIGS. 1 and 2 can include a first connection section 21 which is connected to a urethral catheter or the like, an outer cylinder 22 which is positioned further toward the other end in the axial direction than the first connection section 21, a communicating section 23 which extends in parallel to the axial direction of the outer cylinder 22 from the one end in the axial direction to an outer cylinder opening portion 22a at the other end in the axial direction, in the inside of the outer cylinder 22, and an annular valve body 24 which is provided around the communicating section 23 so as to be movable in the axial direction.

In accordance with an exemplary embodiment, the first connector 2 can include a rigid support member 25, which is provided on a surface of the annular valve body 24 at the one end in the axial direction; and a first elastic body 26, for example, a coil spring which is provided inside the outer cylinder 22 in a posture surrounding the communicating section 23 and biases the annular valve body 24 at all times toward the outer cylinder opening portion 22a through the rigid support member 25. In accordance with an exemplary embodiment, the rigid support member 25 slides and is displaced in the axial direction of the outer cylinder 22 together with the annular valve body 24 in a state where the communicating section 23 is surrounded. Accordingly, an O-ring 25a made of a rubber material or the like can be arbitrarily disposed between the rigid support member 25 and the communicating section 23 in a compressed posture in order to help prevent unintended flow of a fluid between an inner peripheral surface of the rigid support member 25 and an outer peripheral surface of the communicating section 23.

Although not shown in the drawing, the coil spring as the first elastic body 26 can be replaced with an elastic body, for example, a cylindrical rubber member or a tubular rubber member of which the inner and outer surface is formed in a bellows shape, capable of biasing the annular valve body 24 at all times toward the other end of the outer cylinder 22 in the axial direction. In addition, although not shown in the drawing, the first elastic body 26 which is disposed instead of the coil spring can be made as a so-called mechanical spring or the like by being integrally formed with the annular valve body 24 or the rigid support member 25.

As shown in FIGS. 1 and 2, the first connection section 21 is configured to include a first tubular portion 21a which is inserted into an end portion of a tube and has an outer surface formed of a plurality of tapered steps for frictionally engaging the end portion, and a disk shaped portion 21b which has a large outer diameter compared to the first tubular portion 21a. Moreover, a flow path is formed, through which a liquid can circulate, on each of central axes of the portions.

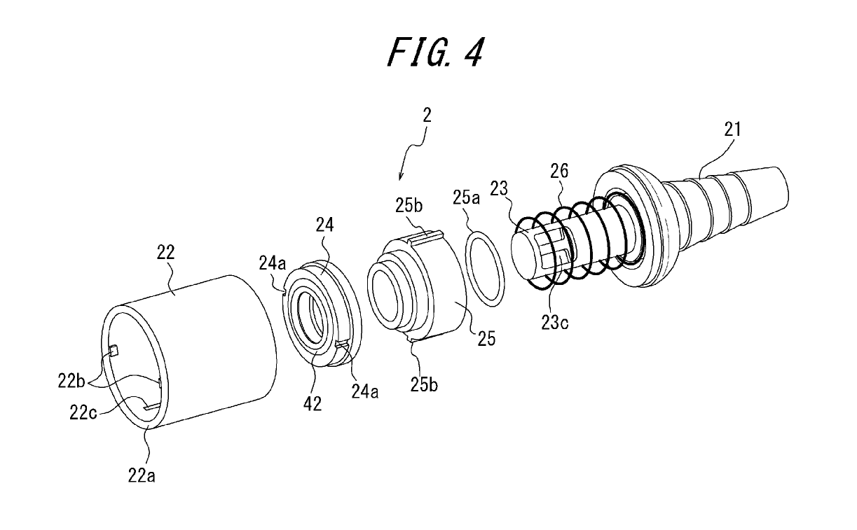

In addition, as shown in FIGS. 1 and 2, the outer cylinder 22 has larger inner and outer diameters than those of the first tubular portion 21a, and has a cylindrical shape with a substantially identical inner diameter (diameter) from the one end of the outer cylinder 22 in the axial direction to the outer cylinder opening portion 22a at the other end in the axial direction. The one end of the outer cylinder 22 in the axial direction is liquid-tightly closed by the disk shaped portion 21b of the first connection section 21. In accordance with an exemplary embodiment, the outer peripheral surface of the outer cylinder 22 shown in FIG. 1, the cross section orthogonal to the axial direction has a circular shape, but the outer peripheral surface of the outer cylinder 22 can be made to have an arbitrary shape. In contrast, as shown in FIG. 4, the inner peripheral surface of the outer cylinder 22 is formed with an inward projection portion 22b, which is used when the second connector 3 is fitted with the first connector 2, to be described later.

In the exemplary embodiment shown in the drawings, the communicating section 23 is attached to the disk shaped portion 21b of the first connection section 21. As shown in FIGS. 1 and 2, the communicating section 23 has a main body portion 23b with a circular tube shape, and a distal portion 23a which seals the other end of the main body portion 23b in the axial direction.

In addition, one or more liquid passages 23c (three liquid passages 23c in the drawing) which extend in the axial direction are formed on a part of a side wall of the main body portion 23b in a circumferential direction. For this reason, a liquid flowing from the first connection section 21 can flow into the communicating section 23 through an opening portion at the one end of the communicating section 23 in the axial direction and can flow out to the outside of the communicating section 23 from the liquid passage 23c. In accordance with an exemplary embodiment, the liquid passage 23c is formed in the other end portion of the side wall of the main body portion 23b in the axial direction. For example, in the fitted state as shown in FIG. 1 in which the second connector 3 is fitted with the first connector 2 to be described in detail later, the liquid passage 23c is formed at a position, at which a liquid can circulate, between one end of the first connection section 21 in the axial direction and a space in the outer cylinder 22 which is located further toward the other end in the axial direction than the other end of the annular valve body 24 in the axial direction, through the liquid passage 23c.

In the non-fitted state in which the second connector 3 is detached from the first connector 2 as shown in FIG. 2, the annular valve body 24 seals the outer cylinder opening portion 22a together with the communicating section 23 and can prevent leakage of a liquid from the inside of the first connector 2 to the other end of the first connector 2 in the axial direction, for example, liquid leakage from the outer cylinder opening portion 22a. For example, in a state where the annular valve body 24 is provided between the communicating section 23 and the outer cylinder 22 and positioned at the outer cylinder opening portion 22a, the annular valve body 24 seals the outer cylinder opening portion 22a together with the distal portion 23a of the communicating section 23 which is positioned inside the annular valve body 24 in a radial direction. In accordance with an exemplary embodiment, when the first elastic body 26 is provided inside the first connector 2, in a state where no input is received from the outside, the annular valve body 24 can be moved to the outer cylinder opening portion 22a by the first elastic body 26 which biases the annular valve body 24 toward the outer cylinder opening portion 22a of the outer cylinder 22 at all times. In accordance with an exemplary embodiment, the annular valve body 24 can be made of, for example, an elastic material.

The second connector 3 shown in FIGS. 1 and 2 can be provided with a second connection section 31, which can be connected to a tube or the like on a urine collection bag between two tubes constituting the urethral catheter, a cylindrical inner cylinder 32 which is positioned further toward the one end in the axial direction than the second connection section 31, and a cap-shaped inner valve body 33 which is provided inside the inner cylinder 32 and on the central axis of the inner cylinder 32 so as to be movable in the axial direction.

Furthermore, the second connector 3 can have a second elastic body 34 such as a coil spring or the like, which biases the inner valve body 33 toward the one end of the inner cylinder 32 in the axial direction at all times, in the inner cylinder 32. In accordance with an exemplary embodiment, the second connector 3 has an elastic body support member 35 which is provided with a columnar projection portion with which the cap-shaped inner valve body 33 is fitted together with the second elastic body 34 in a fitted state, and the second elastic body 34 is provided in a posture surrounding the periphery of the columnar projection portion of the elastic body support member 35. In addition, the cap-shaped inner valve body 33 can have an elastic body accommodation portion 33a, which accommodates the second elastic body 34 in the fitted state shown in FIG. 1, and the second elastic body 34 is disposed in a state where a bottom part of the elastic body accommodation portion 33a and the elastic body support member 35 abut on each other.

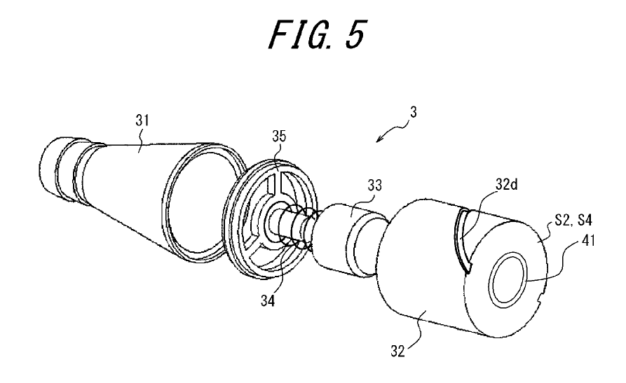

Incidentally, the elastic body support member 35 supporting the second elastic body 34 can be provided between the inner cylinder 32 and a truncated portion 31b of the second connection section 31 as shown in FIG. 5. The elastic body support member 35 can interlock a toric portion and a disk-shaped spring receiving portion, which is disposed inside the toric portion using, for example, three bar-shaped interlock portions at three sites in the circumferential directions thereof, and a member provided with a columnar projection portion can be used in the spring receiving portion, as the elastic body support member.

In the second connector 3, the second elastic body 34 can be accommodated in the elastic body accommodation portion 33a in the fitted state shown in FIG. 1, and therefore, adhesion of a liquid such as urine or the like to the second elastic body 34 can be suppressed by preventing contact between the second elastic body 34 and the liquid flowing in the second connector 3. As a result, urinary tract stones or the like can be prevented from being deposited in the second elastic body 34 or the elastic body accommodation portion 33a.

In accordance with an exemplary embodiment, similar to the above-described first elastic body 26, the coil spring as the second elastic body 34 can be replaced with an elastic body, for example, a cylindrical rubber member or a tubular rubber member of which the inner and outer surface is formed in a bellows shape, capable of biasing the inner valve body 33 toward the one end of the inner cylinder 32 in the axial direction. In addition, although not shown in the drawing, the second elastic body 34 which is disposed instead of the coil spring can be made as a so-called mechanical spring or the like by being integrally formed with the inner valve body 33.

As shown in FIGS. 1 and 2, the second connection section 31 is configured to include a second tubular portion 31a which is inserted into an end portion of a tube and has an outer surface formed of a plurality of tapered steps for frictionally engaging the end portion, and a hollow truncated portion 31b which is positioned between the second tubular portion 31a and the inner cylinder 32 and of which the inner and outer diameters are gradually increased from the second tubular portion 31a toward the inner cylinder 32.

In addition, as shown in FIGS. 1 and 2, the inner cylinder 32 has a cylindrical shape with a slightly smaller outer diameter than the inner diameter of the outer cylinder 22 from the one end of the inner cylinder 32 in the axial direction to the other end of the inner cylinder in the axial direction.

An inner cylinder opening portion 32a which is opened on the central axis of the inner cylinder 32 is provided in one end portion of the inner cylinder 32 in the axial direction. Specifically, the inner cylinder 32 has an annular decreased diameter portion 32b in one end portion thereof in the axial direction, and the inner cylinder opening portion 32a is provided in one end portion of the decreased diameter portion 32b in the axial direction. In addition, the other end portion of the inner cylinder 32 in the axial direction is liquid-tightly closed by the truncated portion 31b of the second connection section 31. In addition, in the inner peripheral surface of the inner cylinder 32, the cross section orthogonal to the axial direction has a circular shape, and the inner peripheral surface of the inner cylinder has a substantially constant inner diameter from the other end of the inner cylinder 32 in the axial direction to the other end of the decreased diameter portion 32b in the axial direction.

Furthermore, the inner diameter of the decreased diameter portion 32b is gradually decreased from the other end in the axial direction toward the one end in the axial direction.

In accordance with an exemplary embodiment, the inner peripheral surface of the inner cylinder 32 can be made to have an arbitrary shape in the cross section orthogonal to the axial direction. In addition, the outer peripheral surface of the inner cylinder 32 can be made to have an arbitrary shape which can be fitted with the outer cylinder 22 of the first connector 2. Furthermore, the inner diameter of the decreased diameter portion 32b may not be gradually decreased as long as the inner diameter of the decreased diameter portion is smaller than that of the other end portion of the inner cylinder 32 in the axial direction, and the decreased diameter portion 32b may have a substantially constant inner diameter.

Furthermore, the inner valve body 33 is provided in the inner cylinder 32 so as to be movable on the central axis of the inner cylinder 32 in the axial direction. The inner valve body 33 can be, for example, made of an elastic material and formed in a cap shape of which the other end in the axial direction is opened. In the fitted state where the second connector 3 is fitted with the first connector 2 as shown in FIG. 1, the inner valve body 33 is positioned inside the inner cylinder 32 by being pushed by the communicating section 23 of the first connector 2 advancing into the inner cylinder 32 through the inner cylinder opening portion 32a, to the other end in the axial direction. In addition, in the non-fitted state where the second connector 3 is detached from the first connector 2 as shown in FIG. 2, the inner valve body 33 is positioned in the inner cylinder opening portion 32a at the one end of the inner cylinder 32 in the axial direction, and the cap-shaped bottom part of the inner valve body seals the one end of the inner cylinder 32 in the axial direction by being fitted with the inner cylinder opening portion 32a. In accordance with an exemplary embodiment, when providing the second elastic body 34 to be described later in the second connector 3, in a state where no input is received from the outside, the inner valve body 33 is moved to the inner cylinder opening portion 32a by the second elastic body 34 which biases the inner valve body 33 toward the inner cylinder opening portion 32a of the inner cylinder 32 at all times.

Such a tube connection structure 1 functions as follows in the fitted state between the first connector 2 and the second connector 3 as shown in FIG. 1 and in the non-fitted state between the first connector 2 and the second connector 3 as shown in FIG. 2.

In the fitted state shown in FIG. 1, the decreased diameter portion 32b in the other end portion of the inner cylinder 32 in the axial direction which enters the outer cylinder 22 pushes the annular valve body 24 to the inside of the outer cylinder 22 against biasing force of the first elastic body 26 while abutting on the annular valve body 24 of the first connector 2, and the communicating section 23 which abuts the inner valve body 33 of the second connector 3 enters the inner cylinder 32 through the inner cylinder opening portion 32a against biasing force of the second elastic body 34 and pushes the inner valve body 33 to the inside of the inner cylinder 32. Accordingly, the annular valve body 24 and the inner valve body 33 are opened, and as shown by arrows in FIG. 1, a liquid from one tube in the axial direction can flow in the outer cylinder 22 and the inner cylinder 32 from the first connection section 21 of the first connector 2 through the liquid passages 23c which are provided on the side wall of the communicating section 23 and can flow in the second connection section 31 of the second connector 3. Accordingly, a liquid can flow from one tube to the other tube through the first connector 2 and the second connector 3.

In contrast, in the non-fitted state shown in FIG. 2, the annular valve body 24 which is pushed to the inside of the outer cylinder 22 by the inner cylinder 32 in the aforesaid fitted state slides and is displaced to the other end of the outer cylinder 22 in the axial direction based on restoring force released from a compressed posture in a case where the first elastic body 26 is provided, due to separation between the first connector 2 and the second connector 3. The outer cylinder opening portion 22a of the outer cylinder 22 is sealed by fitting the annular valve body 24 with the space between the distal portion 23a of the communicating section 23 and the outer cylinder opening portion 22a. In addition, the inner valve body 33 which has been pushed to the inside of the inner cylinder 32 by the communicating section 23 in the aforesaid fitted state is displaced toward the one end of the inner cylinder 32 in the axial direction based on the restoring force released from the compressed posture in a case where the second elastic body 34 is provided. Then, the inner valve body 33 is fitted with the inner cylinder opening portion 32a and the inner cylinder opening portion 32a of the inner cylinder 32 is sealed.

As a result, leakage of a liquid, which flows in each of the tubes can be prevented, from the connectors due to the annular valve body 24 and the inner valve body 33 being closed.

When the above-described first elastic body 26 and the second elastic body 34 are provided, the annular valve body 24 and the inner valve body 33 respectively seal the other end of the outer cylinder 22 in the axial direction and the one end of the inner cylinder 32 in the axial direction due to the biasing force of the first elastic body 26 and the biasing force of the second elastic body 34 in the non-fitted state as shown in FIG. 2 after the first connector 2 is detached from the second connector 3, and therefore, the first connector 2 and the second connector 3 can be used multiple times by repeating the attachment and the detachment.

Accordingly, a urine collection bag can be used by being disconnected from a patient depending on the situation in a case where one end of the tube connection structure 1 is indwelled in the bladder of a patient and the other end of the tube connection structure is provided in the middle of a urethral catheter attached to the urine collection bag. Accordingly, the tube connection structure is suitable to perform so-called rehabilitation, bathing, or the like in a state where a patient is separated from the urine collection bag and to remove any concern of developing urinary tract infection caused by urine flowing backward in the bladder depending on the position or the condition of the urine collection bag.

In accordance with an exemplary embodiment, in a case where these connectors 2 and 3 are used in a state of being fitted together at all times without detaching the first connector 2 from the second connector 3 after the first connector 2 and the second connector 3 are once connected to each other, it is not always necessary to provide the above-described first elastic body 26 and the second elastic body 34, and therefore, although is not shown in the drawing, at least any one of the first elastic body 26 and the second elastic body 34 can be omitted.

In this tube connection structure 1, in order to prevent leakage from the inside of the first connector 2 and the second connector 3 in the fitted state between the first connector 2 and the second connector 3, as shown in FIGS. 1 and 2, the second connector 3 can include a first seal member 41 which is provided at least on the inner peripheral surface of the inner cylinder opening portion 32a and liquid-tightly seals the space between the outer peripheral surface of the communicating section 23 of the first connector 2 inserted into the inner cylinder opening portion 32a and the inner peripheral surface of the inner cylinder opening portion 32a in the fitted state.

In accordance with an exemplary embodiment, the first seal member 41 liquid-tightly seals the space between the outer peripheral surface of the communicating section 23 of the first connector 2 and the inner peripheral surface of the inner cylinder opening portion 32a, and therefore, the liquid passage 23c provided in the communicating section 23 can be disposed at a position entering the inner cylinder 32 in the fitted state.

In accordance with an exemplary embodiment, if the liquid passage 23c is not positioned further toward the other end (inside of the inner cylinder 32) in the axial direction than the inner cylinder opening portion 32a in the fitted state, a liquid cannot circulate through the first connector 2 and the second connector 3.

In accordance with an exemplary embodiment, the first seal member 41 is not particularly limited as long as the space between the outer peripheral surface of the communicating section 23 of the first connector 2 inserted into the inner cylinder opening portion 32a and the inner peripheral surface of the inner cylinder opening portion 32a can be liquid-tightly sealed in the fitted state, and for example, the first seal member 41 can be formed of an elastic member. In addition, the first seal member 41 may be provided on the inner peripheral surface of the inner cylinder opening portion 32a over the entire periphery as shown in FIG. 5, and alternately, the first seal member can be configured so as to seal the inner peripheral surface of the inner cylinder opening portion 32a over the entire periphery by being provided in a part of the inner peripheral surface of the inner cylinder opening portion 32a (not shown) and being crushed when the communicating section 23 is inserted into the inner cylinder opening portion 32a.

According to the tube connection structure 1 of the present disclosure, as shown in FIG. 1, the liquid passage 23c of the communicating section 23 of the first connector 2 is provided in a portion penetrating the inner cylinder opening portion 32a of the second connector 3 and entering the inner cylinder 32 in a fitted state, and the first seal member 41 is provided at least on the inner peripheral surface of the inner cylinder opening portion 32a and liquid-tightly seals the space between the outer peripheral surface of the communicating section 23 of the first connector 2 which is penetrated through the inner cylinder opening portion 32a and the inner peripheral surface of the inner cylinder opening portion 32a in the fitted state. Therefore, leakage of a liquid from the inside of the tube connection structure 1 while making a liquid circulate between one end of the first connector 2 in the axial direction and the other end of the second connector 3 in the axial direction in the fitted state can be prevented. In accordance with an exemplary embodiment, when the liquid passage 23c extends further toward the one end in the axial direction than the inner cylinder opening portion 32a in the fitted state, a flow path of a liquid is formed in the outside of the inner cylinder 32 which is sealed by the first seal member 41, and therefore, there is a concern that a liquid may leak from the abutting surface between the first connector 2 and the second connector 3.

In addition, according to the tube connection structure 1, it is unnecessary to form a concave portion, which can be used for preventing leakage of a liquid in the fitted state and can make it difficult to perform sterilization or to remove a liquid, on an end surface at the other end of the first connector 2 in the axial direction and on an end surface at the one end of the second connector 3 in the axial direction, in the non-fitted state. Therefore, the sterilization of these end surfaces can be easily and reliably performed and a liquid or a foreign substance adhered on the end surfaces can be relatively easily and reliably wiped off. As a result, the tube connection structure 1 of this disclosure can secure a sanitary condition when the first connector 2 and the second connector 3 are connected to each other.

Furthermore, the inner valve body 33 and the inner cylinder opening portion 32a of the second connector 3 can be fitted together by interposing the first seal member 41 in the non-fitted state where the first connector 2 and the second connector 3 are not fitted together. Therefore, the liquid-tightness in the inner cylinder opening portion 32a in the non-fitted state compared to the case where the first seal member 41 is not interposed therebetween can be enhanced. Therefore, for example, the second elastic body 34 having low elastic force compared to a connector without the first seal member 41 can be used, and as a result, a burden of a user when the first connector 2 and the second connector 3 are fitted together can be reduced. In accordance with an exemplary embodiment, when the inner valve body 33 is formed of an elastic material, the inner valve body 33 and the first seal member 41 are favorably stuck to each other, and thus, the liquid-tightness in the inner cylinder opening portion 32a in the non-fitted state can be further enhanced. Therefore, sufficient liquid-tightness can be secured even if the elastic force of the second elastic body 34 is further decreased. Accordingly, the burden of a user when the first connector 2 and the second connector 3 are fitted together can be further reduced.

In accordance with an exemplary embodiment, when attaching and detaching the connectors 2 and 3, for example, when pushing the second connector 3 to the first connector 2 while twisting the second connector, or pulling and detaching the second connector from the first connector 2, the communicating section 23 slides on the surface of the first seal member 41 provided on the inner peripheral surface of the inner cylinder opening portion 32a in the axial direction and the circumferential direction of the inner cylinder 32.

Accordingly, it can be desirable that the first seal member 41 has durability and liquid leakage resistance with respect to the sliding. For example, it can be desirable that the tube connection structure 1, which is utilized in the urethral catheter worn by a patient over a long period of time, has durability and liquid leakage resistance with respect to attachment and detachment between the first connector 2 and the second connector 3 which are repeatedly performed.

Accordingly, it can be preferable that the first seal member 41 is extended from the inner peripheral surface of the inner cylinder opening portion 32a to the inside of the inner cylinder 32 as shown in FIGS. 1 to 3A. For example, it can be preferable that the first seal member 41 is extended to the side of an inner surface 32c of the decreased diameter portion 32b. Accordingly, the adhesion area between the first seal member 41 and the inner cylinder 32 can be increased and the durability of the first seal member 41 by extending the first seal member 41 from the inner peripheral surface of the inner cylinder opening portion 32a to widely provide the first seal member 41 can be enhanced. As a result, liquid leakage resistance can also be enhanced

In addition, in a case where the inner diameter of the decreased diameter portion 32b of the inner cylinder 32 is gradually decreased from the other end in the axial direction toward the one end in the axial direction as shown in FIGS. 1 to 3A, it can be preferable that the range of the first seal member 41 to be extended is set to be within a range R further toward the one end of the decreased diameter portion 32b in the axial direction than the other end of the decreased diameter portion in the axial direction from the inner peripheral surface of the inner cylinder opening portion 32a, and it is more preferable that the first seal member 41 extends to the other end of the decreased diameter portion 32b in the axial direction as shown in the drawings. Accordingly, durability of the first seal member 41 with respect to the sliding during the attachment and detachment of the connectors 2 and 3 can be enhanced, by widely providing the first seal member 41 as described above. As a result, liquid leakage resistance can also be enhanced.

In accordance with an exemplary embodiment, the cross-sectional area of the inside of the inner cylinder 32 further toward the other end of the inside of the inner cylinder 32 in the axial direction than the decreased diameter portion 32b is greater than that of the decreased diameter portion 32b, and therefore, the flow rate of a liquid circulating therethrough deteriorates. Accordingly, when the tube connection structure 1 is applied to the urethral catheter, for example, solid bodies such as urinary tract stones or the like discharged from the inside of the body of a patient through the urethral catheter easily remain further toward the other end in the axial direction than the decreased diameter portion 32b. In contrast, when the first seal member 41 is extended from the inner peripheral surface of the inner cylinder opening portion 32a to the other end of the inner cylinder opening portion in the axial direction exceeding the other end of the decreased diameter portion 32b in the axial direction, the first seal member 41 disturbs the circulation of a liquid, of which the flow rate is decreased further toward the other end in the axial direction than the decreased diameter portion 32b, and therefore, urinary tract stones or the like discharged from the inside of the body more easily remain in the inner cylinder 32. Accordingly, it can be preferable that the first seal member 41 is installed within the range R further toward the one end of the decreased diameter portion 32b in the axial direction than the other end of the decreased diameter portion in the axial direction. Moreover, it can be more preferable that the first seal member 41 extends to the other end of the decreased diameter portion 32b in the axial direction in view of enhancing durability and liquid leakage resistance of the first seal member 41 while suppressing the remaining of the urinary tract stones or the like.

In accordance with an exemplary embodiment, in the tube connection structure 1, the first seal member 41 preferably extends from the inner peripheral surface of the inner cylinder opening portion 32a onto the end surface at the one end of the inner cylinder 32 in the axial direction. For example, in the views shown in FIGS. 1 to 3A, the first seal member 41 extends onto the end surface at the one end of the decreased diameter portion 32b in the axial direction. Accordingly, the adhesion area between the first seal member 41 and the inner cylinder 32 can be increased and the durability of the first seal member 41 can be enhanced with respect to the sliding during the attachment and detachment of the connectors by extending the first seal member 41 onto the end surface at the one end of the inner cylinder 32 in the axial direction to widely provide the first seal member. As a result, liquid leakage resistance cam be improved as well. In addition, with the first seal member 41 extending onto the end surface at the one end of the inner cylinder 32 in the axial direction, it can be possible to more efficiently wipe off a liquid, which has adhered around the communicating section 23 entering the inner cylinder 32, using the first seal member 41 abutting on the communicating section 23 when the structure enters the non-fitted state from the fitted state. Furthermore, since the first seal member 41, which extends onto the end surface of the inner cylinder 32, abuts on the annular valve body 24 of the first connector 2 in the fitted state, sealing properties in a portion where the annular valve body 24 and the end surface of the inner cylinder 32 abut on each other can be enhanced. Furthermore, in a case where the first seal member 41 is not extended onto the end surface of the inner cylinder 32 and is provided only in a portion from the inner peripheral surface of the inner cylinder opening portion 32a to the other end of the decreased diameter portion 32b in the axial direction, it can be difficult to sufficiently secure sealing properties while a portion of the communicating section 23 of the first connector 2 which is formed with the liquid passage 23c passes through the inner cylinder opening portion 32a. However, in the configuration in which the first seal member 41 extends onto the end surface of the inner cylinder 32, the first seal member 41, which extends onto the end surface, abuts on the annular valve body 24 before the annular valve body 24 and the inner valve body 33 are opened when the first connector 2 and the second connector 3 are fitted together. Accordingly, liquid-tightness in the connection section between the first connector 2 and the second connector 3 during the connection can be sufficiently secured.

In accordance with an exemplary embodiment, it can be particularly preferable that the first seal member 41 is disposed from the inner surface 32c of the inner cylinder 32 onto the end surface at the one end of the inner cylinder 32 in the axial direction through the inner peripheral surface of the inner cylinder opening portion 32a as shown in FIGS. 1 to 3A, which can enhance durability with respect to the sliding of the inner cylinder 32 in the circumferential direction if the first seal member 41 is extended to both sides of the inner peripheral surface of the inner cylinder opening portion 32a and the decreased diameter portion 32a is interposed between the first seal members 41 in the axial direction.

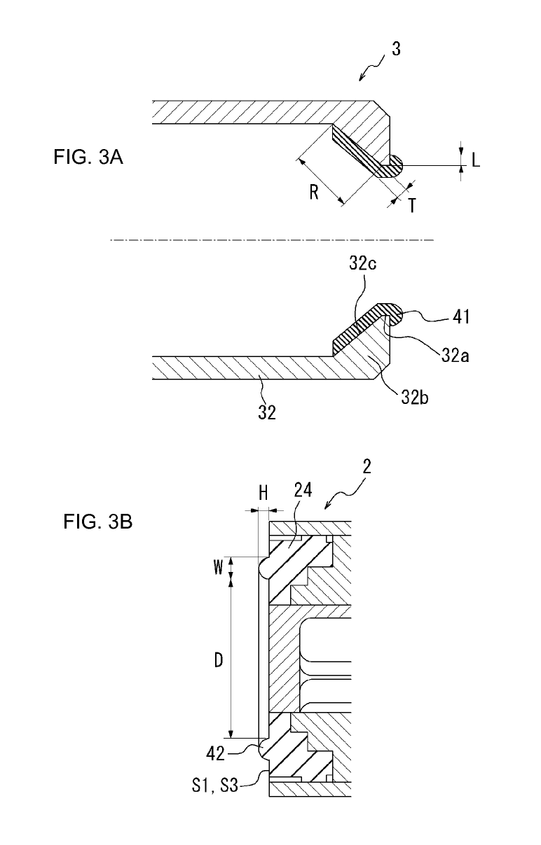

In accordance with an exemplary embodiment, the thickness T of the first seal member 41 provided in the inner cylinder opening portion 32a as shown in FIG. 3A is preferably within the range of 0.5 mm to 1.0 mm. In accordance with an exemplary embodiment, a sanitary condition of the end surface of the second connector 3 can be maintained while securing sealing properties in the inner cylinder opening portion 32a by setting the thickness T of the first seal member 41 to a thickness within the aforesaid range.

In accordance with an exemplary embodiment, when the thickness T of the first seal member 41 is set to be less than 0.5 mm, there can be a concern that the sealing properties may not be secured and the durability of the first seal member 41 may be decreased. In addition, in a case where the thickness T is set to be greater than 1.0 mm, for example, when the first seal member 41 extends onto the end surface at the one end of the inner cylinder 32 in the axial direction, there can be a concern that it may be difficult to wipe off a liquid or the like adhered on the end surface of the second connector 3 in the non-fitted state, and therefore, wiping residues may be particularly generated in a fine portion around the first seal member 41.

As shown in FIG. 3A, in a case where the first seal member 41 extends from the inner peripheral surface of the inner cylinder opening portion 32a onto the end surface at the one end of the inner cylinder 32 in the axial direction, the length L of the first seal member 41 which is measured along a radial direction from the inner peripheral surface of the inner cylinder opening portion 32a to an outer end of the first seal member 41 in the radial direction which is positioned on the end surface is preferably within a range of 0.5 mm to 1.0 mm, which can help guarantee pressure resistance and remove wiping residues due to cleaning by setting the length L of the first seal member 41 to be within the aforesaid range.

In accordance with an exemplary embodiment, when the length L of the first seal member 41 is set to be shorter than 0.5 mm, there can be a concern that the pressure resistance may not be guaranteed. In addition, when the length L exceeds 1.0 mm, there can be a concern that wiping residues due to cleaning may be generated.

In accordance with an exemplary embodiment, the attachment of the first seal member 41 to the inner cylinder 32 can be achieved by attaching the first seal member 41, which has been separately molded, to the inside of the inner cylinder 32, which has been previously molded, using a well-known adhesive or through fusion or the like. In addition, the attachment of the first seal member 41 to the inner cylinder 32 can be achieved by forming the inner cylinder 32 and the first seal member 41 through two-color molding, using a material constituting the inner cylinder 32 and a material constituting the first seal member 41.

As shown in FIGS. 1 and 2, in the tube connection structure 1, when the first connector 2 and the second connector 3 are fitted together, an annular second seal member 42 is preferably provided on at least one of opposing surfaces including an opposing surface S1 of the annular valve body 24 of the first connector 2 which is opposite to the end surface at the one end of the inner cylinder 32 of the second connector 3 in the axial direction, and an opposing surface S2 on the end surface of the one end of the inner cylinder 32 in the axial direction which is opposite to the annular valve body 24, so as to protrude from the one opposing surface and abut on the other opposing surface in the fitted state. In accordance with an exemplary embodiment, the second seal member 42 can be on the opposing surface S1 of the annular valve body 24 of the first connector 2. Accordingly, the second seal member 42 abutting on the other opposing surface over the entire periphery in the fitted state can help reliably seals the abutting portion of the second seal member 42 in addition to helping the leakage prevention due to the above-described first seal member 41. Accordingly, a liquid can be reliably prevented from leaking to the outside of the second seal member 42 even when the first seal member 41 is twisted or plastically deformed by repeatedly inserting and removing the communicating section 23 with respect to the inner cylinder 32, for example. Furthermore, it can be difficult to sufficiently secure the sealing properties while the portion of the communicating section 23, which is formed with the liquid passage 23c, passes through the inner cylinder opening portion 32a, only using the first seal member 41. However, in this configuration, the second seal member 42 abuts on the opposing surface before the annular valve body 24 and the inner valve body 33 are opened when the first connector 2 and the second connector 3 are fitted together. Accordingly, liquid-tightness in the connection section between the first connector 2 and the second connector 3 during the connection can be sufficiently secured. In accordance with an exemplary embodiment, the configuration in which the liquid-tightness between the connectors during the connection is secured using the second seal member 42 can be, for example, useful when the first seal member 41 is not extended onto the end surface of the inner cylinder 32 and is provided only at the other end of the decreased diameter portion 32b in the axial direction from the inner peripheral surface of the inner cylinder opening portion 32a.

In accordance with an exemplary embodiment, the second seal member 42 can be integrally formed with the opposing surface to be disposed, or can be attached to the opposing surface, to be disposed, for example, using a well-known adhesive or through fusion or the like by making it as a separate ring-shaped member. In this manner, the second seal member 42 can be formed as a member different from the annular valve body 24 or the inner cylinder 32 using a desired material regardless of the materials of the annular valve body 24 and the inner cylinder 32, and accordingly, for example, sealing properties as expected using the second seal member 42 can be exhibited.

In addition, for example, the aforesaid second seal member 42 can be provided on the opposing surface S1 of the annular valve body 24 of the first connector 2.

Furthermore, the second seal member 42 provided on an opposing surface and the other opposing surface on which the second seal member 42 abuts in the fitted state can be formed of elastic materials or rigid materials, and alternately, one of the second seal member and the other opposing surface can be formed of an elastic material and the other one can be formed of a rigid material, as long as a seal between the second seal member and the opposing surface can be secured. However, when the second seal member 42 is formed on the opposing surface S1 of the annular valve body 24, it is preferable to form the second seal member 42 with an elastic material and form the opposing surface S2 of the inner cylinder 32 abutting on the second seal member 42 with a rigid material in the fitted state. Accordingly, the second seal member 42, which is formed of an elastic material, abuts on and is crushed by the opposing surface S2 of the inner cylinder 32, which is formed of a rigid material, in the fitted state, and is more reliably sealed by the abutting portion of the second seal member 42, and therefore, effectively preventing a liquid from leaking to the outside of the second seal member 42, which can be necessary for the inner cylinder 32 to have rigidity to some extent since the inner cylinder 32 is used when pushing the annular valve body 24 to the inside of the outer cylinder 22.

In the tube connection structure 1, surface areas S3 and S4 other than the portions, on the end surface at the other end of the first connector 2 in the axial direction and the end surface at the one end of the second connector 3 in the axial direction, in which the first seal member 41 and the second seal member 42 are positioned, for example, can be made to be flat surfaces in the non-fitted state where the first connector 2 and the second connector 3 are not fitted together.

For example, in the first connector 2, it can be preferable that the surface portion except for an area on the opposing surface S1 of the annular valve body 24 which is positioned in the outer cylinder opening portion 22a of the outer cylinder 22, the area being formed with the second seal member 42 in the case where the second seal member is formed, and the outer surface of the distal portion 23a of the communicating section 23 are positioned on a substantially identical plane in the non-fitted state shown in FIG. 2. Accordingly the end surface of the first connector 2, which is formed by the opposing surface S1 of the annular valve body 24 positioned in the outer cylinder opening portion 22a of the outer cylinder 22 and the outer surface of the distal portion 23a of the communicating section 23 can be made to be a flat surface in a portion (surface area S3) other than the second seal member 42. In addition, in the second connector 3, it can be preferable that the outer surface of the inner valve body 33 fitted into the inner cylinder opening portion 32a and the surface area except for an area (both areas formed with the first seal member 41 and the second seal member 42 when the second seal member 42 is formed on the opposing surface S2) formed with the first seal member 41 on the opposing surface S2 of the inner cylinder 32 are positioned on a substantially identical plane in the non-fitted state shown in FIG. 2. Accordingly, the end surface of the second connector 3, which is formed by the outer surface of the inner valve body 33 at the one end of the inner cylinder 32 in the axial direction and the opposing surface S2 of the inner cylinder 32, can be a flat surface in a portion (surface area S4) other than the first seal member 41 (both the first seal member 41 and the second seal member 42 when the second seal member 42 is formed on the opposing surface S2).

In accordance with an exemplary embodiment, there is no concave portion recessed inward, as in the related art, on both of the end surfaces of the first connector 2 and the second connector 3 which are formed as described above. Therefore, after the first connector 2 can be detached from the second connector 3 and the state of the tube connection structure is set to the non-fitted state shown in FIG. 2, a liquid or a foreign substance adhered on the end surfaces of these connectors can be easily and reliably wiped out using a fabric or the like without any residues. Subsequently, i the end surfaces of the connectors can be relatively easily and reliably sterilization by applying a drug.

Furthermore, in the tube connection structure 1, both of the end surfaces of the first connector 2 and the second connector 3 are not positioned deeply inside the outer cylinder 22 or the inner cylinder 32 and are exposed at the other end of the outer cylinder 22 in the axial direction or at the one end of the inner cylinder 32 in the axial direction, in the non-fitted state shown in FIG. 2. Therefore, removal of a liquid adhered on the end surfaces and sterilization of the end surfaces can be more easily and reliably performed.

From this point of view, it can be preferable that the surface area S3 on the end surface of the first connector 2 is positioned on a substantially identical plane with the end surface of the outer cylinder 22 at the other end as shown in FIG. 2.

In accordance with an exemplary embodiment, as shown in FIG. 3B, which is an enlarged cross-sectional view of the first connector 2 in a non-fitted state, it can be preferable that the protrusion height H from the opposing surface of the second seal member 42 is a height at which the second seal member 42 disappears by abutting on and being crushed by the opposing surface in the fitted state between the first connector 2 and the second connector 3, the height specifically being 0.5 mm to 1.5 mm. Accordingly, the opposing surface of the annular valve body 24 and the opposing surface of the inner cylinder 32 more approach each other as they are brought into contact with each other in a fitted state, and therefore, the sealing properties using the second seal member 42 can be greatly enhanced.

In accordance with an exemplary embodiment, when the protrusion height H is set to be less than 0.5 mm, there can be a concern that leakage of a liquid may be caused in the fitted state between the first connector 2 and the second connector 3. In addition, when the protrusion height H exceeds 1.5 mm, it can be difficult to wipe off a liquid or the like adhered on the end surface formed with the second seal member, and therefore, wiping residues may be generated especially in a fine portion in the inside and the outside of the second seal member 42 in the radial direction.

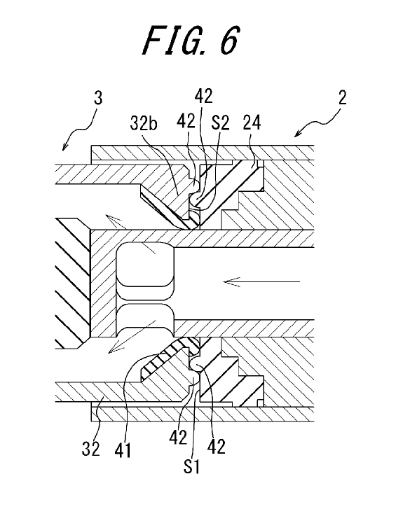

FIG. 3B shows a form in which the second seal member 42 is provided on the annular valve body 24 of the first connector 2. However, as described above, the second seal member 42 can be provided on the inner cylinder 32 of the second connector 3, and second seal members 42 can be provided on both of the annular valve body 24 of the first connector 2 and the inner cylinder 32 of the second connector 3 as shown in FIG. 6. In accordance with an exemplary embodiment, when the second seal member 42 is provided on both of the annular valve body 24 and the inner cylinder 32, each of the second seal members 42 may be disposed at positions separated from each other in a fitted state, or may be disposed at positions at which the second seal members 42 are abutted with each other in a fitted state, for example, at positions at which the second seal member 42 on the annular valve body 24 side and the second seal member 42 on the inner cylinder 32 side face each other. In accordance with an exemplary embodiment, it can be preferable that the second seal members 42 are provided at positions abutting with each other in a fitted state as shown in FIG. 6 from the viewpoint of sufficiently improving the sealing property by the second seal member 42.

The width W of the second seal member 42 can be preferably set to 0.5 mm to 1.5 mm. When the width W is less than 0.5 mm, there can be a concern that the sealing properties cannot be sufficiently secured. In contrast, when the width W is greater than 1.5 mm, there can be a concern that a level difference of the second seal member 42 in the surface portion around the second seal member may become large, and therefore, it can be impossible to obtain sufficient benefits with easiness of wiping off of a liquid resulted from making the end surface be a flat surface.

In addition, the inner diameter D of the second seal member 42 is preferably set to 8 mm to 10 mm. When the inner diameter D of the second seal member 42 is less than 8 mm, the surface portion surrounded by the second seal member 42 becomes narrow, and therefore, it can be difficult to wipe the portion with, for example, the thumb. In addition, when the inner diameter D exceeds 10 mm, the distance between the outer peripheral edge of the end surface of the connector and the second seal member 42 becomes shorter, and therefore, it can be difficult to wipe the space between the outer peripheral edge and the second seal member 42.

In accordance with an exemplary embodiment, although the second seal member 42 is made to have a toric shape in the embodiment in FIG. 1 or the like, the second seal member 42 is not limited to the form shown in the drawing as long as the shape of the second seal member is a shape which can surround the inner cylinder opening portion 32a in the fitted state shown in FIG. 1 and seal the inner cylinder opening portion 32a.

In accordance with an exemplary embodiment, when the second seal member 42 is provided on the opposing surface of the first connector 2 in addition to the first seal member 41, it can be preferable that the second seal member 42 is disposed in a form in which the second seal member 42 surrounds the first seal member 41 by being brought into contact therewith in a fitted state, such that the pressure resistance is maintained since the seal members 41 and 42 are compressed or crushed.

Moreover, in order to fix the first connector 2 and the second connector 3 to each other in a state where the first connector 2 and the second connector 3 are fitted together, for example, one or greater inward projection portions 22b, preferably two inward projection portions 22b facing each other, which protrude toward the inside of the outer cylinder 22 can be provided on the inner peripheral surface of the outer cylinder opening portion 22a of the first connector 2 as shown in FIG. 4, and two guide groove portions 32d in this specification which open at the one end of the inner cylinder 32 in the axial direction and extend from the one end of the inner cylinder 32 toward the other end in the axial direction of the inner cylinder are provided on the outer peripheral surface of the inner cylinder 32 of the second connector 3 in accordance with the disposed positions or the number of inward projection portions 22b as shown in FIG. 5.

In accordance with an exemplary embodiment, when the above-described inward projection portions 22b are provided in the outer cylinder opening portion 22a of the first connector 2, concave portions 24a of the annular valve body 24 can be provided, which can be positioned in the outer cylinder opening portion 22a using the biasing force of the first elastic body 26 in a non-fitted state between the first connector 2 and the second connector 3 and is used for entering the inward projection portions 22b as shown in FIG. 4, in a portion corresponding to the disposed positions of the inward projection portions 22b in the circumferential direction. The surface portion of the annular valve body 24 can be made to be positioned on a substantially identical plane with the end surface at the other end of the outer cylinder 22 in the axial direction in a non-fitted state as shown in FIG. 2 by providing the concave portion 24a and further providing the inward projection portion 22b protruding inward in the outer cylinder opening portion 22a of the outer cylinder 22.

In addition, linear grooves 22c, for example, two linear grooves (only the lower side linear groove 22c is shown in FIG. 4), extending parallel to the axial direction of the outer cylinder 22 as shown in FIG. 4, can be provided on the inner surface of the outer cylinder 22 and fitting convex portions 25b, which extend parallel to the axial direction of the outer cylinder 22, are fitted in the linear grooves, and slide in the linear grooves 22c, can be provided at positions on the outer peripheral surface of the rigid support member 25 which respectively correspond to the linear grooves 22c in the circumferential direction, in order to make displacement of the annular valve body 24, which is performed together with the rigid support member 25 in the outer cylinder 22 and is caused by contraction and restoration of the first elastic body 26, be always performed parallel to the axial direction of the outer cylinder 22 and in an identical posture with respect to the outer cylinder 22.

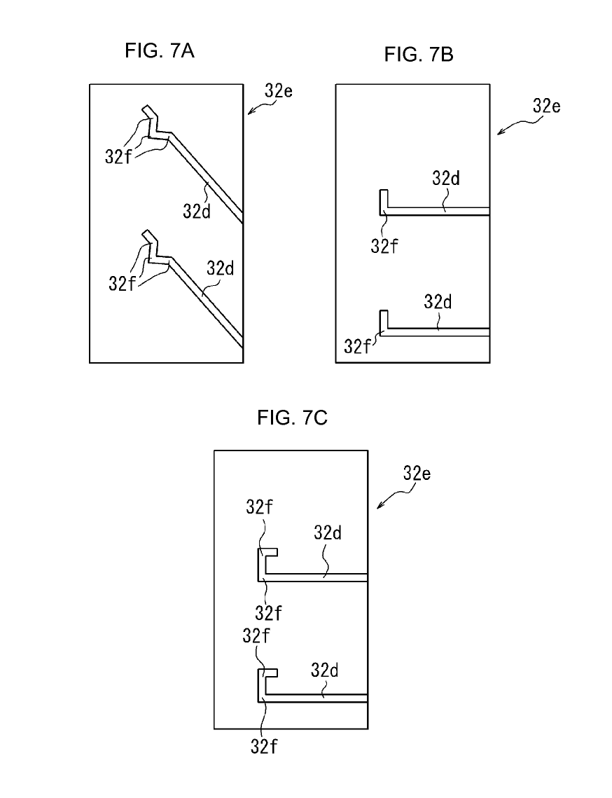

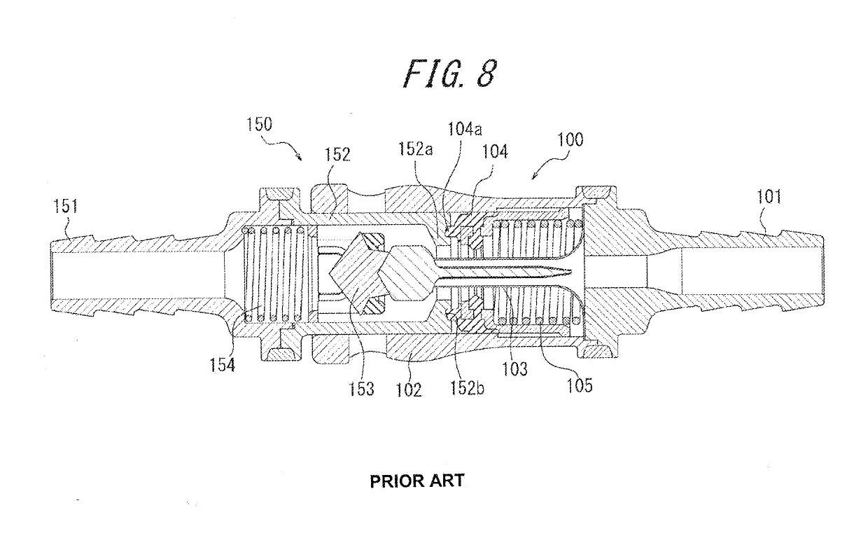

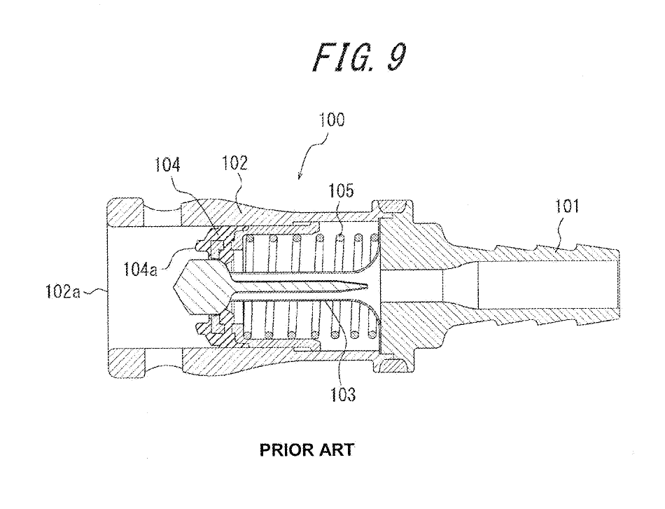

Here, as shown in FIG. 7A is a view of an outer peripheral surface 32e of the inner cylinder 32, the guide groove portion 32d shown in FIG. 5 extends in a posture inclined to the axial direction of the inner cylinder 32 along the outer peripheral surface 32e of the inner cylinder 32 from the one end (right side in FIGS. 7A-7C) of the inner cylinder 32 in the axial direction to the other end (left side in FIGS. 7A-7C) of the inner cylinder in the axial direction with a width slightly wider than that of the inward projection portion 22b over the hole length. Moreover, the end portion at the other end of the guide groove portion 32d in the axial direction can be formed with three bent sections 32f and is bent into a convex mountain shape toward the other end of the inner cylinder 32 in the axial direction.

In a case where such an inward projection portion 22b and a guide groove portion 32d are formed, fixing of the first connector 2 and the second connector 3 to each other in a fitted state can be achieved by making the inward projection portion 22b climb the mountain-shaped bent sections 32f of the guide groove portion 32d under slightly large amount of screwing force applied to the outer cylinder 22 when the outer cylinder 22 is screwed with respect to the inner cylinder 32 and the inward projection portion 22b reaches the bent sections 32f of the guide groove portion 32d in order to make the inward projection portion 22b slide from the one end of the inner cylinder 32 in the axial direction toward the other end of the inner cylinder in the corresponding guide groove portion 32d. In accordance with an exemplary embodiment, when the connectors 2 and 3 having such an inward projection portion 22b and a guide groove portion 32d enter a fitted state, the guide groove portion 32d is formed in a posture inclined in the axial direction of the inner cylinder 32, and therefore, a user can make the connectors enter the fitted state with weak force compared to when the guide groove portion 32d is formed in a posture parallel to the axial direction of the inner cylinder 32.

In the guide groove portion 32d, a click sound can be generated when the inward projection portion 22b climbs the bent sections 32f, and therefore, a user can relatively easily recognize that the first connector 2 and the second connector 3 are fixed to each other. In addition, the bent sections 32f also function to help prevent the first connector 2 from unintentionally coming off from the second connector 3 and lock them to each other.

In contrast, in the guide groove portion 32d, the second connector 3 can be set to be come off from the first connector 2 when a certain degree of tensile force is applied to a tube. Therefore, in a case where the tube junction structure is provided in the midway of the urethral catheter as described above, the second connector 3 can be set to be come off from the first connector 2 when a certain degree of tensile force is applied to the tube. Accordingly, when the tube is pulled with a large amount of force, the urethral catheter can be avoided, which is indwelled in the bladder of a patient, from being pulled from the bladder without coming off of the second connector 3 from the first connector 2.

In accordance with an exemplary embodiment, the guide groove portion 32d can have various extending forms including a form in which the guide groove portion 32d extends parallel to the axial direction of the inner cylinder 32 and is bent in one side in the circumferential direction (vertical direction in FIGS. 7A-7C) of the inner cylinder 32, for example, bent at right angle, at the bent section 32f formed at the other end of the inner cylinder 32 in the axial direction as shown in FIG. 7B instead of the guide groove portion 32d shown in FIG. 7A; a form in which the guide groove portion 32d extends parallel to the axial direction of the inner cylinder 32 and is bent in one side in the circumferential direction of the inner cylinder 32 at the bent section 32f similarly to the view shown in FIG. 7B, and then, is further bent at the bent section 32f, slightly extends to the one end of the inner cylinder 32 in the axial direction, and ends as shown in FIG. 7C.

When the guide groove portions 32d shown in FIGS. 7B and 7C are provided, the guide groove portions can be respectively fixed by frictionally engaging the inward projection portion 22b onto a groove wall surface of the portion of the guide groove portion 32d which extends in the circumferential direction of the inner cylinder 32 in the form shown in FIG. 7C and by fitting the inward projection portion to the portion of the guide groove portion 32d which extends to the other end of the inner cylinder 32 in the form shown in FIG. 7C based on restoring force of the first elastic body 26 and the second elastic body 34 in the axial direction which are contracted, by relatively rotating the first connector 2 and the second connector 3 when the inward projection portion 22b is made to slide from the one end to the other end of the inner cylinder 32 in the axial direction in the guide groove portion 32d by pushing the first connector 2 to the second connector 3 without twisting the first connector and reaches the bent section 32f in the circumferential direction of the inner cylinder 32.

In addition, in the guide groove portions 32d shown in FIGS. 7B and 7C, the first connector 2 can be pushed into the second connector 3 without being twisted to slide the inward projection portion 22b at the bent sections 32f. Accordingly, torsion of the first seal member 41 due to the communicating section 23 can be suppressed when these connectors are in the fitted state compared to the guide groove portion 32d shown in FIG. 7A. Therefore, deterioration in the first seal member 41 due to attachment and detachment repeated in the first connector 2 and the second connector 3 can be suppressed.

In accordance with an exemplary embodiment, in all of the above-described guide groove portions 32d, the aforesaid bent sections can be set to curved sections not shown in the drawing.

In accordance with an exemplary embodiment, there is no portion protruded outside the tube connection structure 1 in such a locking mechanism constituted by the inward projection portion 22b and the guide groove portion 32d, and therefore, even if the tube connection structure 1 comes into contact with the skin of a patient, the tube connection structure 1 does not damage the skin, and therefore, it is particularly efficient when the tube connection structure is used in a medical and welfare tube.

Hereinabove, the embodiment of the present disclosure has been described with reference to accompanying drawings, but the tube connection structure of the present invention is not limited to the aforesaid examples and can be appropriately modified.

According to the present disclosure, a tube connection structure is disclosed which makes it possible to easily and reliably perform sterilization or the like of a first connector and a second connector through application of a drug before connecting the connectors to each other and can be used in a sanitary condition.

The detailed description above describes a tube connection structure. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A tube connection structure, used in a living body, comprising:
a first connector which has a first connection section connected to a tube in one end portion in an axial direction;
a second connector which has a second connection section connected to a tube in a other end portion in the axial direction and of which the one end portion in the axial direction is fitted with the other end portion of the first connector in the axial direction, wherein in a fitted state between the first connector and the second connector, a liquid can circulate between one end of the first connector in the axial direction and a other end of the second connector in the axial direction through the inside of the fitted first and second connectors, and in a non-fitted state between the first connector and the second connector, leakage of a liquid from the inside of the first connector to the other end of the first connector in the axial direction and leakage of a liquid from the inside of the second connector to one end of the second connector in the axial direction are prevented;
the first connector includes an outer cylinder which is positioned further toward the other end in the axial direction than the first connection section;
a communicating section which extends from the one end in the axial direction to an outer cylinder opening portion at the other end in the axial direction in the outer cylinder and is formed with a liquid passage in which the liquid can circulate in the fitted state;
an annular valve body which is provided around the communicating section so as to be movable in the axial direction and seals the outer cylinder opening portion at the other end of the outer cylinder in the axial direction in the non-fitted state;
the second connector includes an inner cylinder which is positioned further toward the one end in the axial direction than the second connection section and pushes the annular valve body to one side in the axial direction by entering the outer cylinder in the fitted state; an inner valve body which is provided in the inner cylinder so as to be movable in the axial direction, seals one end of the inner cylinder in the axial direction by being fitted with an inner cylinder opening portion of the one end of the inner cylinder in the axial direction in the non-fitted state and is pushed to the other end in the axial direction by the communicating section of the first connector advancing into the inner cylinder through the inner cylinder opening portion in the fitted state; and a first seal member which is provided at least on an inner peripheral surface of the inner cylinder opening portion and liquid-tightly seals the space between an outer peripheral surface of the communicating section of the first connector which is penetrated through the inner cylinder opening portion and the inner peripheral surface of the inner cylinder opening portion in the fitted state; and
the liquid passage of the communicating section is provided in a portion entering the inner cylinder in the fitted state.

2. The tube connection structure according to claim 1, wherein
the inner cylinder has a decreased diameter portion, which is provided with the inner cylinder opening portion and of which the inner diameter is decreased, in the one end portion in the axial direction, and
the first seal member extends from the inner peripheral surface of the inner cylinder opening portion to an inner surface side of the decreased diameter portion.

3. The tube connection structure according to claim 2, wherein
the inner diameter of the decreased diameter portion is gradually decreased from the other end in the axial direction to the one end in the axial direction, and
the first seal member extends to the other end of the decreased diameter portion in the axial direction.

4. The tube connection structure according to claim 1, wherein
the first seal member extends onto an end surface at the one end of the inner cylinder in the axial direction.

5. The tube connection structure according to claim 1, wherein
an annular second seal portion is provided on at least one of opposing surfaces counter to the annular valve body and the inner cylinder, so as to protrude from the one opposing surface and abut on the other opposing surface in the fitted state.

6. The tube connection structure according to claim 1, wherein
the first connector further includes a first elastic body which biases the annular valve body toward the other end of the outer cylinder in the axial direction of the outer cylinder, and the second connector further includes a second elastic body which biases the inner valve body toward one side in the axial direction of the inner cylinder.

7. The tube connection structure according to claim 2, wherein
the first seal member extends onto an end surface at the one end of the inner cylinder in the axial direction.

8. The tube connection structure according to claim 3, wherein
the first seal member extends onto an end surface at the one end of the inner cylinder in the axial direction.

9. The tube connection structure according to claim 2, wherein
an annular second seal portion is provided on at least one of opposing surfaces counter to the annular valve body and the inner cylinder, so as to protrude from the one opposing surface and abut on the other opposing surface in the fitted state.

10. The tube connection structure according to claim 3, wherein
an annular second seal portion is provided on at least one of opposing surfaces counter to the annular valve body and the inner cylinder, so as to protrude from the one opposing surface and abut on the other opposing surface in the fitted state.

11. The tube connection structure according to claim 4, wherein
an annular second seal portion is provided on at least one of opposing surfaces counter to the annular valve body and the inner cylinder, so as to protrude from the one opposing surface and abut on the other opposing surface in the fitted state.

12. The tube connection structure according to claim 2, wherein
the first connector further includes a first elastic body which biases the annular valve body toward the other end of the outer cylinder in the axial direction of the outer cylinder, and the second connector further includes a second elastic body which biases the inner valve body toward one side in the axial direction of the inner cylinder.

13. The tube connection structure according to claim 3, wherein
the first connector further includes a first elastic body which biases the annular valve body toward the other end of the outer cylinder in the axial direction of the outer cylinder, and the second connector further includes a second elastic body which biases the inner valve body toward one side in the axial direction of the inner cylinder.

14. The tube connection structure according to claim 4, wherein
the first connector further includes a first elastic body which biases the annular valve body toward the other end of the outer cylinder in the axial direction of the outer cylinder, and the second connector further includes a second elastic body which biases the inner valve body toward one side in the axial direction of the inner cylinder.

15. The tube connection structure according to claim 5, wherein
the first connector further includes a first elastic body which biases the annular valve body toward the other end of the outer cylinder in the axial direction of the outer cylinder, and the second connector further includes a second elastic body which biases the inner valve body toward one side in the axial direction of the inner cylinder.
